(19) United States
(12) Patent Application Publication
(10) Pub. No.: US 2019/0309345 A1
(43) Pub. Date: Oct. 10, 2019

(54) CLINICAL APPLICATION OF CELL FREE DNA TECHNOLOGIES TO NON-INVASIVE PRENATAL DIAGNOSIS AND OTHER LIQUID BIOPSIES

(71) Applicants: Baylor College of Medicine, Houston, TX (US); Baylor Miraca Genetics Laboratories, LLC, Houston, TX (US)

(72) Inventors: Lee-Jun C. Wong, Sugar Land, TX (US); Jinglan Zhang, Houston, TX (US); Jianli Li, Houston, TX (US); Yanming Feng, Houston, TX (US); Arthur L. Beaudet, Houston, TX (US); Hongzheng Dai, Houston, TX (US); Xiaoyan Ge, Houston, TX (US); Hui Mei, Houston, TX (US); Guoli Wang, Houston, TX (US)

(21) Appl. No.: 16/331,112

(22) PCT Filed: Sep. 7, 2017
(86) PCT No.: PCT/US17/50510

§ 371 Date: Mar. 6, 2019

Related U.S. Application Data
(60) Provisional application No. 62/384,282, filed on Sep. 7, 2016.

(51) Int. Cl. C12Q 1/6806; C40B 50/06; C40B 40/08; C12Q 1/6827; C12Q 1/6855; C12Q 1/6883

(52) U.S. Cl. CPC C12Q 1/6806; C40B 50/06; C40B 40/08; C12Q 1/6827; C12Q 1/6855; C12Q 2565/514; C12Q 2525/161; C12Q 2525/191; C12Q 2535/122; C12Q 2537/159; C12Q 2563/179; C12Q 1/6883

(57) ABSTRACT

Embodiments of the disclosure include methods of prenatal testing using non-invasive means that identify single gene disorders. In specific embodiments the methods are non-invasive and employ tagging circulating cell-free fetal DNA from the biological mother with particular adaptors that employ unique barcodes, followed by steps to enrich targets and steps for thorough sequencing.

### CROSS-REFERENCE TO RELATED APPLICATIONS

The present application claims priority to U.S. Provisional Patent Application Ser. No. 62/384,282, filed Sep. 7, 2016, which is incorporated by reference herein in its entirety.

### TECHNICAL FIELD

The field of the disclosure generally includes at least the fields of cell biology, molecular biology, diagnostics, bioinformatics, nucleic acid processing, and medicine.

### BACKGROUND

Since the discovery of fetal cell-free DNA (cfDNA) in the late 1990s, noninvasive prenatal testing (NIPT) for genetic diseases has advanced tremendously and become a practical screening test for some chromosomal aneuploidies (1, 2). Especially with the advent of massively parallel sequencing or next-generation sequencing (NGS), millions of NIPT tests were conducted worldwide in the last few years. The NGS based NIPT demonstrated much improved test sensitivity and specificity compared to traditional maternal serum screening for trisomy 21, 18 and 13(1). NIPT also became increasingly available for sex chromosome aneuploidies and subchromosomal deletions/duplications although its clinical utility for these conditions remain to be proven (3-5). Besides chromosomal or subchromosomal copy number changes, non-invasive prenatal diagnosis for Mendelian disorders was developed, but was clinically available at a much smaller scale. Such tests were only applicable to a limited number of genes or known familial pathogenic variants using digital PCR, Sanger sequencing or NGS on a targeted basis for genes or variants in question (6-8). Although these methods demonstrated satisfactory results to detect genetic defects in a small targeted region, they are difficult to be used as a high-throughput screening test for a broad spectrum of Mendelian diseases. It has been shown that cfDNA can be used to construct an entire fetal genome (9). Therefore, it is plausible to utilize cfDNA to detect essentially all genetic aberrations in fetal genome including small genetic alterations (e.g., single nucleotide variants, small insertions or deletions and etc.). A recent study demonstrated improved analytical performance of NIPT to detect pathogenic variants in single genes using a PCR-free library construction coupled with high-coverage whole genome sequencing, but it is still cost prohibitive to be used as a screening test for a large population (10). Besides the logistics constraints, the low fraction of pathogenic variants present in the fetal and maternal cfDNA admixture imposes a great challenge to develop wet lab procedures and bioinformatics pipeline to detect and interpret such genetic changes in a clinical setting (10).

The present application satisfies a long-felt need in the art to provide efficient useful methods for fetal DNA analysis.

### BRIEF SUMMARY

The present invention is directed to methods and compositions for analysis of fetal DNA. In particular embodiments, the methods are non-invasive and utilize analysis of cell-free DNA, including circulating cell-free DNA. In particular embodiments methods of the disclosure are for identifying variants associated with single gene disorders, although other types of disorders may be identified.

In certain embodiments, the present disclosure concerns the development, validation and early clinical implementation of the first non-invasive prenatal screening test on circulating cell-free DNA (cfDNA) in maternal blood for de novo or paternally inherited pathogenic variants (as examples only) in a variety of genes frequently associated with dominant monogenic diseases.

In one embodiment, there is a non-invasive method of analyzing fetal DNA for one or more variants therein, comprising the steps of: (a1) generating or providing a collection of circulating cell-free fetal DNA (cfDNA) fragments, each fragment comprising a first end ligated to a first adaptor and a second end ligated to a second adaptor, to produce fetal adaptor-ligated molecules, wherein the first adaptor comprises a first strand and second strand having a complementary region there between that comprises a unique barcode and wherein the second adaptor comprises a first strand and second strand having a complementary region there between that comprises a unique barcode; and (a2) generating or providing a collection of DNA fragments from the biological mother of the fetus and/or a separate collection of DNA fragments from the biological father of the fetus, wherein the fragments in the collection(s) comprise adaptor-ligated ends to produce maternal adaptor-ligated molecules and paternal adaptor-ligated molecules, respectively, wherein the adaptors each comprise a first strand and second strand having a complementary region there between; (b) amplifying the fetal, maternal, and paternal adaptor-ligated molecules with primers complementary to a region of the respective adaptors to produce amplified adaptor-ligated molecules; (c) enriching the amplified adaptor-ligated molecules for one or more target sequences of interest to produce enriched adaptor-ligated molecules; (d) amplifying the enriched adaptor-ligated molecules; (e) sequencing at least some of the enriched adaptor-ligated molecules; and (f) analyzing the sequenced enriched adaptor-ligated molecules. In specific embodiments, the adaptors in step (a2) lack a unique barcode. In particular embodiments, in step (b) a primer binds a region of the respective adaptor-ligated molecules and/or in step (b) a primer binds the fetal adaptor-ligated molecules at a region that is 5′ to the unique barcode.

In specific embodiments, first and second adaptors each comprise a 5′ single-stranded end on their respective first strands in relation to their respective 3′ ends of their respective second strands. In certain cases, the unique barcode comprises 6 or more random nucleotides.

In particular cases, the cfDNA fragments and/or the DNA fragments from the biological mother and biological father are subjected to end repair of the fragments and tailing of the fragment ends with a known nucleotide that is complementary to a nucleotide on the 3′ ends of the first strands of the adaptors. The collection of DNA fragments from the biological mother and biological father are produced by fragmentation of genomic DNA from the biological mother and biological father, respectively, in at least some cases.

In certain cases, the enriching step comprises exposing the amplified adaptor-ligated molecules to probes that hybridize to a region of the amplified adaptor-ligated molecules, and the probes may target coding sequence. The probes may be linked to a directly detectable agent or indirectly detectable agent. The probes may be linked to a first binding agent that binds to a second binding agent, and in specific cases the first binding agent is biotin and the second binding agent is avidin. The second binding agent may or may not be linked to a substrate, such as a bead, plate, column, or well. The target sequence may be a coding sequence of a gene, including a coding sequence for an exon. In specific embodiments, the target sequence(s) are of one or more genes associated with a monogenic Mendelian disorder. The target sequences of interest may be sequences from one or more genes in a collection of genes. The collection may be a collection of sequences from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more different genes. The sequencing for the method may be of any kind, including next generation sequencing.

In at least some cases, an analyzing step comprises comparing sequence between fetal sequenced enriched adaptor-ligated molecules and maternal sequenced enriched adaptor-ligated molecules and/or paternal sequenced enriched adaptor-ligated molecules.

In particular embodiments, the fetal DNA is obtained from the blood or plasma of the biological mother. In some cases DNA is obtained from biopsies, such as liquid biopsies, such as for the detection of cancer.

Variants for the fetal DNA may be one or more de novo variants or are paternally-inherited. The variant may be a single point mutation, insertion, deletion, or inversion. The biological age of the father may be 45 years or greater. The variant may be associated with a monogenic Mendelian disorder. In some cases, the DNA of the biological father has a known variant associated with a disorder. The variant may not be aneuploidy, in some cases.

In certain embodiments the method further comprises the step of assaying a fetal sample using an invasive and/or postnatal method for the fetus and/or biological mother. In some cases, at least one assay for the fetus during gestation had a determination of an abnormality or had a determination of a suspected abnormality

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1C show an example and testing of a workflow for one embodiment of a method of the disclosure. In FIG. 1A, there is an illustration of adaptor ligation to cell-free DNA. In FIG. 1B, there is determination of the number of duplicate reads for adaptor-ligated cell-free DNA molecules. In FIG. 1C, there is measurement of the reduction of errors introduced upon library and sequencing steps of embodiments of the method.

FIGS. 2A-2B show estimations for fetal fractions. In FIG. 2A, there is determination of fetal fraction estimate upon comparison of a SNP-based determination from NGS reads on a particular example of a gene, SRY. In FIG. 2B, fetal fraction as a function of gestational age is determined.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

The present disclosure concerns methods, compositions, and systems for determining from a sample whether there is the presence of disease or the risk of disease. In particular, the disclosure concerns non-invasive methods for testing a sample for the presence of disease or risk thereof. In specific cases the sample is from a pregnant female wherein the sample comprises fetal DNA and a risk for disease for the fetus is determined, for example. Embodiments of the disclosure include prenatal testing, and the testing may be routine for an individual or the testing may be because of a risk for an individual having a disease (for example, individuals with a family history). In at least some cases, a determination of an abnormal pregnancy or risk thereof or suspicion thereof has been identified. In specific embodiments, sample(s) from a pregnant individual are subjected to methods of the disclosure to determine the presence of disease or a risk thereof in the fetus or fetuses.

In specific embodiments, the disclosure concerns non-invasive prenatal testing for a risk of disease in a fetus, and in certain aspects the risk is a result of the presence of one or more genetic mutations (which may be referred to as a variant) associated with a disease. The variant may be of any kind, including a point mutation, deletion, insertion, inversion, combination thereof, and so forth. In specific embodiments, the variant is not aneuploidy.

In specific embodiments, the methods of the disclosure identify fetal genetic variants, whether or not they are at low frequency. The methods of the disclosure are able to distinguish low level variants from sequencing errors of the nucleic acid, in specific embodiments. The variants may be de novo or paternally-inherited, for example.

Although in specific embodiments the maternal sample comprising the fetal cell-free DNA is of any kind, in specific embodiments the sample is from a pregnant biological mother and in particular embodiments the sample comprises cell-free DNA, including cell-free DNA of the fetus that is circulating cell-free DNA in the blood of the biological mother. The circulating cell-free DNA may be obtained from a sample by any suitable method. The circulating cell-free DNA may be obtained with commercial reagents, such as from Qiagen® (Hilden, Germany) or Promega® (Madison, Wis.), for example.

The tissue source of the sample from the biological mother may be of any kind, but in specific embodiments the source is blood, plasma, amniotic fluid, cerebrospinal fluid, nipple aspirate, and so forth.

In cases wherein methods of the disclosure are employed for prenatal testing, the testing may occur at any time during gestation of the fetus, including at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more weeks of gestation. The testing may occur in the first trimester, second trimester, and/or third trimester. In specific cases, the gestational age of the fetus is in the range of 8-22, 8-21, 8-20, 8-19, 8-18, 8-17, 8-16, 8-15, 8-14, 8-13, 8-12, 8-11, 8-10, 8-9, 9-22, 9- 21, 9-20, 9-19, 9-18, 9-17, 9-16, 9-15, 9-14, 9-13, 9-12, 9-11, 9-10, 10-22, 10-21, 10-20, 10-19, 10-18, 10-17, 10-16, 10-15, 10-14, 10-13, 10-12, 10-11, 11-22, 11-21, 11-20, 11-19, 11-18, 11-17, 11-16, 11-15, 11-14, 11-13, 11-12, 12-22, 12-21, 12-20, 12-19, 12-18, 12-17, 12-16, 12-15, 12-14, 12-13, 13-22, 13-21, 13-20, 13-19, 13-18, 13-17, 13-16, 13-15, 13-14, 14-22, 14-21, 14-20, 14-19, 14-18, 14-17, 14-16, 14-15, 15-22, 15-21, 15-20, 15-19, 15-18, 15-17, 15-16, 16-22, 16-21, 16-20, 16-19, 16-18, 16-17, 17-22, 17-21, 17-20, 17-19, 17-18, 18-22, 18-21, 18-20, 18-19, 19-22, 19-21, 19-20, 20-22, 20-21, or 21-22 weeks.

Although any type of disease or risk thereof may be tested for using methods of the disclosure, in specific embodiments the disease is a monogenic disease, including a de novo dominant monogenic disease.

In specific embodiments, the pregnancy is a singleton pregnancy. In at least some cases, the pregnancy is not an abnormal pregnancy and the method involves routine prenatal testing. In certain cases, the biological mother and/or biological father have no known personal or family history of a genetic disorder, although in other cases one or both of the biological parents have a personal or family history of a genetic disorder.

Embodiments of the disclosure include methods for population-based screening, including for single gene Mendelian diseases, as an example.

Embodiments of the disclosure include methods for non-invasive prenatal testing methods to detect de novo mutations in cell-free DNA.

Specific embodiments of the disclosure include non-invasive prenatal testing of single gene disorders for pregnancies with abnormal ultrasound findings and/or advanced paternal age.

The methods of the disclosure may be utilized for any type of individual, including mammals such as humans, dogs, cats, horses, sheep, goats, pigs, and so forth.

In some cases, one or more additional methods are utilized in conjunction with the methods of the present disclosure to confirm the outcome of methods of the disclosure, and such methods may or may not include invasive testing, ultrasound screening, clinical evaluations, MRI, CT, X-ray, a combination thereof, and so forth. In some cases, Sanger sequencing of a particular sequence of interest is used to confirm the outcome of methods of the disclosure.

In specific embodiments, methods of the disclosure test for genetic abnormalities associated diseases in which symptoms are detectable at birth and/or manifest after birth, and the symptoms may be physical, mental, intellectual, or a combination thereof.

In specific embodiments, the fetus for which the testing is being performed has a biological father of a certain age, such as over 40, 41, 42, 43, 44, 45, 46, 47, 48, 49, or 50 years of age or more, for example.

In specific embodiments, the fetus for which the testing is being performed has a biological mother of a certain age, such as over 35, 36, 37, 38, 39, 40, 41, 42, 43, 44, 45, 46, 47, 48, 49, or 50 years of age or more, for example.

### I. Embodiments of Methods of the Disclosure

Generally, embodiments of the disclosure utilize molecular barcoding of cfDNA, target enrichment by hybridization, algorithm(s) for DNA deduplication, and variant calling for next generation sequencing (NGS). In general embodiments, the method encompasses a means for tagging fetal cfDNA using detectable label(s), amplifying the tagged molecules, enriching the amplified molecules, amplifying the enriched amplified molecules, sequencing them, and analyzing the sequence(s).

Embodiments of the methods of the disclosure utilize comparison of fetal DNA sequence with biological father and/or biological mother DNA sequence, although in specific embodiments, the methods occur without comparison of fetal DNA sequence with biological father and/or biological mother DNA sequence.

Preparation of a Collection of DNA

In the methods of the disclosure, fetal cfDNA is prepared for analysis and DNA from the biological mother and/or biological father are prepared for analysis. In at least specific cases, preparation of fetal cfDNA is non-identical to preparation of parental DNA. The fetal cfDNA is fragmented naturally, so no further fragmentation is required. However, the ends of the fragmented fetal cfDNA must be prepared such that adaptors can be ligated thereto. Therefore, the ends may be polished using a polymerase (for example, Taq polymerase), and in specific embodiments a single nucleotide is added to the ends of the fragments to facilitate ligation of the adaptors thereto.

Parental DNA collections may be prepared by obtaining the parental DNA from the biological mother and biological father of the fetus. In specific embodiments, the parental DNA is genomic and must be manipulated to be smaller, more manageable fragments. Therefore, in specific embodiments the parental genomic DNA is fragmented, such as by shearing or enzyme digestion, and a certain range of sizes (for example, 100-500 bp) may be isolated for further use in the methods of the disclosure.

Prior to subsequent steps, the fetal and/or parental DNA may be quantified.

Adaptor Ligation and Adaptors

Following preparation of the collection(s) of DNA, adaptors are ligated to the DNA fragments, such as to facilitate subsequent amplification of the DNA fragments. In specific embodiments, the adaptors may be Y-shaped, and in some cases they may be commercially obtained. In certain embodiments, adaptors that are ligated to fetal DNA fragments are not from the same population of adaptors that are ligated to parental DNA fragments.

In specific embodiments, one or more particular adaptors are utilized in methods of the disclosure. In particular embodiments, adaptors are utilized for ligating to cfDNA such that the cfDNA may be individually and uniquely labeled. In specific embodiments the adaptors are ligated to cfDNA so that fragments of cfDNA may be individually labeled with molecular barcodes. In specific embodiments, the adaptors are ligated to cfDNA so that each fragment is labeled with a molecular barcode on each end of the cfDNA fragment. In certain cases, the adaptors used for ligation to parental DNA fragments do not comprise a barcode.

In specific embodiments, molecular barcodes in the adaptors comprise one or more particular features. For example, each molecular barcode may comprise a certain number of random bases, and in specific embodiments the number of random bases in the barcode is a value that allows for the corresponding adaptor-labeled fragment to be uniquely labeled, such as at least 5, 6, 7, 8, 9, 10, 11, 12, or more random bases.

In specific embodiments, an adaptor comprises a first and a second strand that comprise complementarity there between. Thus, in specific cases, part of the adaptor may be double stranded and part of the adaptor may be single stranded. When the first and second strand are hybridized together, in specific embodiments there may be a 5′ extension on at least one strand. In a first strand in a 5′ to 3′ direction, there may be a region that comprises sequence that is not complementary to the 3′ end of the corresponding second strand. The first strand comprises a molecular barcode comprising a number of random bases, such as 5-12 random bases, or more. In a region of the first strand that is 3′ on the strand in relation to the molecular barcode, there may be a specific number of nucleotides having a known sequence, such as 3, 4, 5, or more nucleotides. The 3′ end of the first strand may comprise a particular nucleotide, including one that is complementary to a nucleotide on the ends of cfDNA (for example when the cf DNA is tailed with a particular nucleotide). The second strand of the adaptor may comprise sequence that is complementary to the specific number of nucleotides on the corresponding first strand. In a position that is 3′ on the second strand to this, there may be a molecular barcode that is complementary in sequence to the molecular barcode on the first strand. The 3′ end of the second strand may comprise sequence that is not complementary to the first strand. In specific embodiments, a known, unique sequence (that may be referred to as universal) is present on the adaptor such that the universal sequence may be targeted upon amplification. The universal sequence may be of any suitable length and/or content.

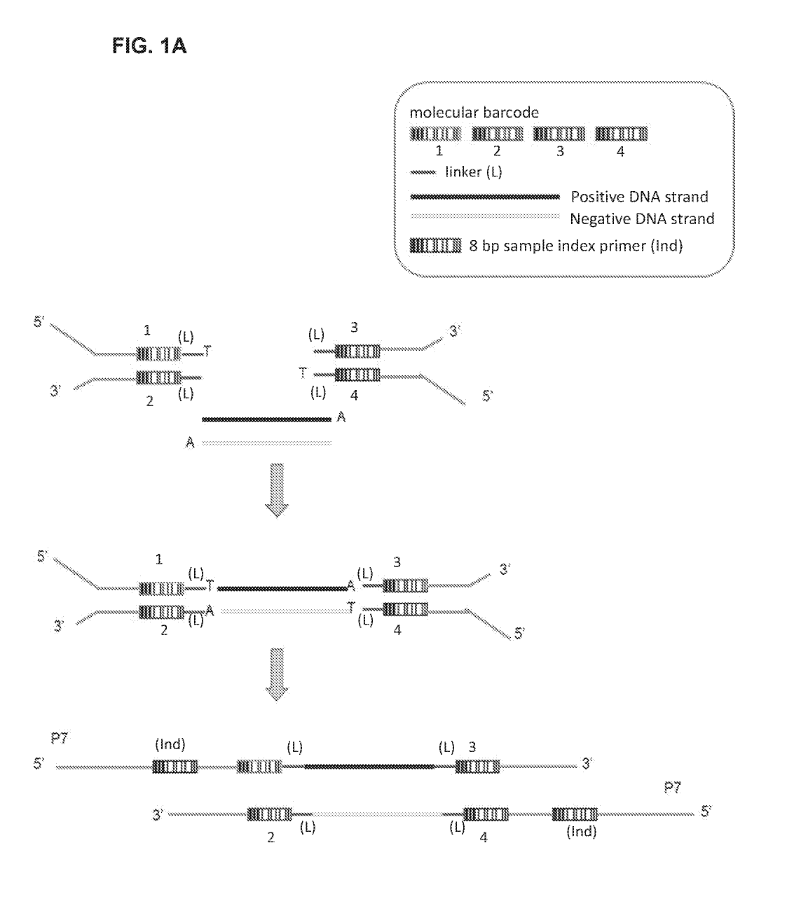

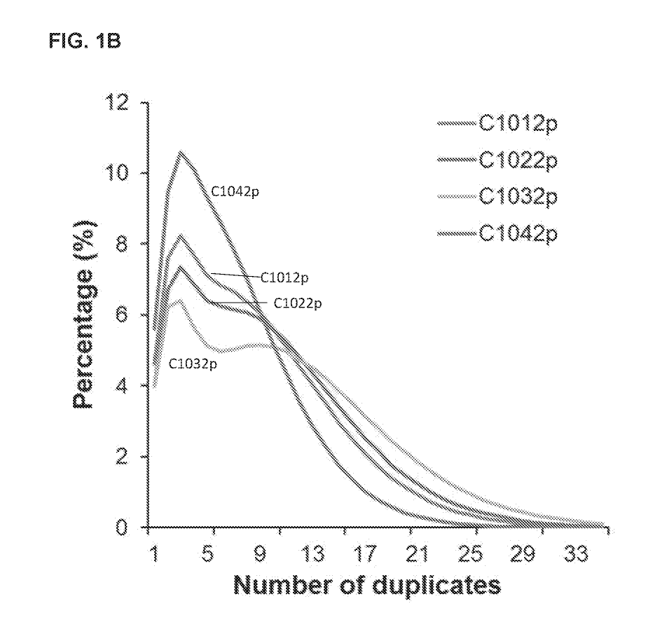

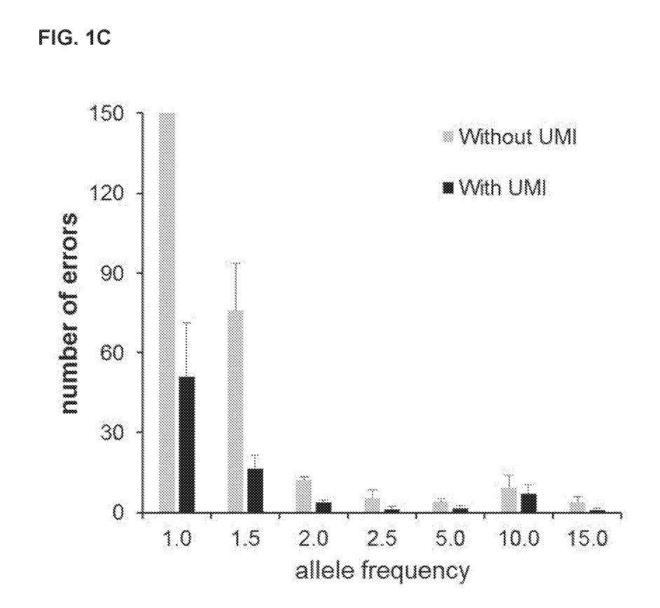

Ligation of the adaptors to the fragments may occur by suitable means known in the art but in specific embodiments occurs through single complementary nucleotides on the 3′ ends of first strands of the adaptor to the corresponding end of the DNA fragment (see FIG. 1). In specific embodiments, the single complementary nucleotides are A/T.

Amplification and Enrichment of Adaptor-Ligated Molecules

Once the adaptors have been ligated to the fetal cfDNA fragments and, when appropriate, the parental DNA fragments, the adaptor-ligated molecules may be amplified, such as to produce a suitable amount of material to be utilized in subsequent steps. In specific embodiments, primers that target the adaptor-ligated molecules are utilized in any suitable types of amplification. In specific cases, primers that target a universal sequence that is common to at least some of the adaptor-ligated molecules are utilized. In particular embodiments, the primers target a universal sequence that on the adaptors for fetal cfDNA fragments is 5′ to the molecular barcode region of the adaptor. In specific embodiments, amplification occurs by PCR.

Once the adaptor-ligated molecules have been amplified, in particular embodiments a collection of amplified adaptor-ligated molecules having specific sequence(s) of interest are enriched to facilitate analysis of only desired sequence(s) of interest. In specific embodiments, such an enrichment step comprises isolation of the desired amplified adaptor-ligated molecules away from amplified adaptor-ligated molecules that lack sequence(s) of interest. In specific embodiments, the enrichment occurs using hybridization. For example, oligonucleotide probes of known sequence may be exposed in parallel to the separate collections of fetal adaptor-ligated molecules, maternal adaptor-ligated molecules, and paternal adaptor-ligated molecules (in cases wherein fetal DNA is compared to parental DNA). The probes may be of any suitable length such that they are able to recognize a particular desired sequence. In specific cases the probes target particular regions of genes of interest, such as genes that are associated with monogenic Mendelian disorders. In certain cases the probes target exon coding sequences.

In particular embodiments, the probes are labeled. The probes may be linked to a first binding agent that binds to a second binding agent, and this binding may be exploited to allow isolation of the adaptor-ligated molecules that comprise sequence(s) of interest. In specific embodiments, the first and second binding agents are biotin and avidin, respectively. Thus, in certain cases the probes are labeled with biotin and the biotin-labeled probes bind avidin, and upon this binding the desired adaptor-ligated molecules are isolated from the collection. In specific embodiments, the second binding agent (such as avidin) is linked to a substrate that facilitates the isolation, such as a bead, plate, well, and so forth. The bound substrate may be washed with suitable buffer(s) to remove the undesired adaptor-ligated molecules.

In specific embodiments, the isolated adaptor-ligated molecules are amplified to provide suitable amount of nucleic acid for subsequent steps in the method.

Sequencing and Analysis of the Enriched Adaptor-Ligated Molecules

In certain embodiments, the enriched adaptor-ligated molecules are sequenced and analyzed for variants. The sequence of the fetal enriched adaptor-ligated molecules may be compared to the sequence of the maternal enriched adaptor-ligated molecules and/or paternal enriched adaptor-ligated molecules, including sequence of one or more specific sequences in one or more different genes.

In specific embodiments, the sequencing is high throughput sequencing, such as next generation sequencing (NGS).

Once at least part of the sequence of the enriched adaptor-ligated molecules is determined, the sequence may be analyzed for the presence of one or more variants. This analysis may or may not comprise direct comparison for the absence or presence of the same variants in the maternal enriched adaptor-ligated molecules and/or paternal enriched adaptor-ligated molecules. In some cases, mathematical computations are utilized as part of the analysis, such as algorithms.

The target sequences that are analyzed may be of any kind, but in specific embodiments the target sequences that are analyzed are from different genes. In certain embodiments, one or more sequences are analyzed from one gene and one or more sequences are analyzed from one or more different genes. The number of genes having sequence to be analyzed may be of any number, including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, or more different genes. Any gene may be analyzed, and the skilled artisan recognizes which genes are associated with certain genetic diseases, for example based on databases such as Online Mendelian Inheritance in Man (OMIM).

Once a variant has been identified in a fetal sample, action may be taken to treat or lessen the severity of the disease associated with the variant, for example. Personalized medicine may be provided to the fetus, and correction of the genetic defect may occur either in utero or postnatal.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Testing of Fetal DNA for Variants

The incidence of single gene disorders in live-born individuals is ˜0.36%, while the aggregated incidence of chromosomal anomalies is 0.18%. Yet, current non-invasive prenatal testing (NIPT) is targeted towards detection of chromosomal abnormalities in the fetus, while a prenatal screening test for pathogenic variants in multiple single genes is not available.

Methods

Plasma sample of 170 pregnant women and 47 spike-in samples with known pathogenic variants were used in this example of a study. After tagging cfDNA with unique molecular index by adaptor ligation and hybridization-based target enrichment followed by next-generation sequencing, the target region was analyzed with average read-depth of >1,000×. A set of regions containing 153 highly polymorphic SNPs were used to determine fetal fraction. All positive results were confirmed by a secondary assay and/or Sanger sequencing on DNA from invasive or postnatal specimens.

Findings

Positive results were reported for 14 pregnant women from 142 eligible participants (9.9%), which included 11 de novo and 3 paternally-inherited pathogenic variants. These pathogenic variants including those in COL1A1, COL1A2, FGFR3, NIPBL, PTPN11 and RIT1 (as examples) were successfully identified and confirmed in pregnancies with abnormal ultrasound findings or known paternal history of conditions included in the screening panel.

Significance of Certain Embodiments

Described herein is a highly sensitive and specific non-invasive prenatal screening method for de novo or paternally-inherited pathogenic variants in maternal blood. The test demonstrates its usefulness for pregnancies with abnormal ultrasound findings or positive paternal history in the related genes, as examples. Clinical studies on larger numbers of samples from pregnant women may be performed to evaluate the clinical performance of this new test which is useful as a population-based non-invasive prenatal screening for single gene Mendelian disorders caused by de novo mutations, and in the setting of advanced paternal age as an extension of NWT for aneuploidy, for example.

### Example 2

### Non-Invasive Prenatal Testing of Single Gene Disorders for Pregnancies with Abnormal Ultrasound Findings or Advanced Paternal Age

The Application of Unique Molecular Indexing to Suppress Sequencing Artifacts

During the PCR of NGS library construction and sequencing processes, random DNA changes can be introduced that result in an increase in sequence background noise (11-14). The test specificity is exacerbated for the detection of de novo or paternal alleles in maternal cfDNA that are usually present in low percentages in maternal plasma cfDNA. To aid in separating the bona fide variants from artifacts, unique molecular indexing (UMI) is used in the library construction process. The UMI used in this assay comprises degenerated nucleotides and a linker of fixed sequences (FIG. 1A). Because the UMIs are at least 10⁵ times of the numbers of DNA molecules in 10 ng gDNA input, these DNA fragments can be individually labeled with different UMIs even though some fetal cfDNA fragments may have identical 5′ and 3′ ends (10). The number of reads with the same UMI is an important indicator to determine whether the sequencing is deep enough to capture essentially all distinct input DNA molecules. It was ensured that >90% of each UMI-labeled molecule has at least two reads (FIG. 1B). Next, the errors introduced during NGS library preparation were examined, and sequencing steps were reduced by ˜9 folds for the variants with allele frequency less than 1% (FIG. 1C). The low percentage calls that only appeared in the plasma samples and not in both parents are considered as errors since the de novo rate is 1.20×10⁻⁸ per nucleotide per generation. The systematic errors or platform dependent errors are further suppressed by a custom database with accumulation of all recurrent calls.

Estimation of Fetal Fraction

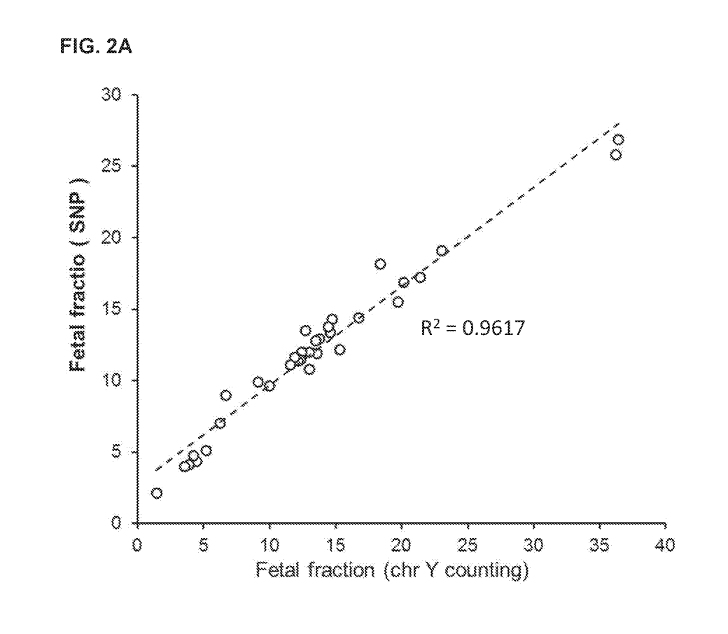

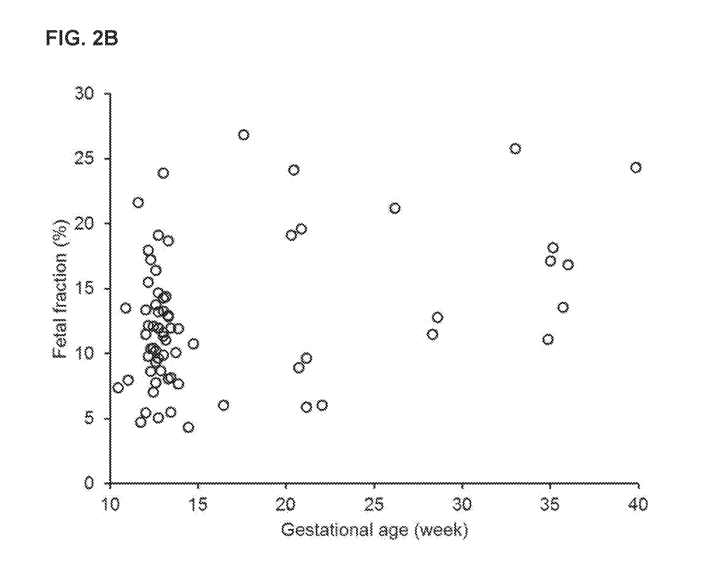

A component of NIPT is to determine the fetal fraction (FF) of total cell-free DNA extracted from maternal plasma as it affects the test sensitivity of the assay, in specific embodiments. Tared the performance of a SNP-based FF calculation was compared with the results obtained through NGS reads containing SRY gene on 33 male fetuses. These two methods yielded consistent results (R2=0.962) for FF estimate (FIG. 2A). The estimation of FF is shown in FIG. 2B. The median of FF is 11.4% (2.2 to 36.9%), similar to a previous report (15). Additionally, the analytical validity of this new SNP based FF calculation method was verified by spike-in studies which were conducted using extracted DNA from a proband sample and genomic DNA from the proband's mother and father. DNA from each proband sample was added to maternal DNA to achieve final concentrations of the proband DNA ranging from 1 to 20%. A strong correlation (R2=0.97) was observed between the calculated fetal fraction and the estimated fetal fraction based on the percentage of proband DNA in the corresponding maternal DNA.

Test Sensitivity, Specificity and Reproducibility

The study design for determining the accuracy, sensitivity, and specificity was based on the detection of cell-free DNA changes in plasma DNA and parental genomic DNA. In 47 spike-in samples with DNA ranging from 2.5% to 10%, the expected frequencies of heterozygous mutations are about half of spike-in percentage. All pathogenic mutations with expected frequencies were detected (Table 1), including single point mutations and small indels such as SMC1A:c.802_804del (p.K268del) and COL1A1:c.3709_3716delAGCCTGAG (p.S1237fs).

TABLE 1
Detection of Pathogenic Variants in Spike-In Samples

| Gene | Pathogenic variant | 2.5% spike-in | coverage | 5% spike-in | coverage | 10% spike-in | coverage |
|---|---|---|---|---|---|---|---|
| SYNGAP1 | c.3190C > T | 1.00 | 1004 | | | | |
| KRAS | c.458A > T | 0.70 | 862 | 2.05 | 996 | | |
| NRAS | c.35G > A | 1.71 | 776 | 2.06 | 1118 | | |
| TSC2 | c.1864C > T | 1.33 | 1357 | 2.39 | 1214 | | |
| SHOC2 | c.4A > G | 1.38 | 796 | 2.92 | 788 | 4.08 | 735 |
| SMC1A | c.802_804del | 1.48 | 1351 | 2.14 | 1074 | 4.98 | 1425 |
| COL1A1 | c.3709_3716del | 0.77 | 1176 | 1.36 | 1252 | 4.01 | 1148 |
| SOS1 | c.508A > G | 0.59 | 505 | 2.68 | 598 | 5.82 | 395 |
| PTPN11 | c.1505C > T | 0.72 | 692 | 1.49 | 536 | 3.95 | 532 |
| MECP2 | c.806delG | | | 2.22 | 857 | 3.91 | 920 |
| CDKL5 | c.2701dupC | | | 2.50 | 679 | 3.43 | 729 |
| CHD7 | c.3379-1G > A | | | 1.58 | 498 | 5.49 | 565 |
| FGFR3 | c.1620C > A | | | 1.96 | 815 | | |

In Table 1, the gray regions represent DNA that was limited in certain samples and not all concentrations could be tested.

In the validation study, 47 trios from 76 pregnant women were recruited, which included two women with clinical or family history related to conditions screened by this test and 45 women without target conditions. One de novo pathogenic variant, c.1138G>A (p.G380R) was identified in FGFR3 gene of sample P1, which demonstrated short femur length (<3%) during ultrasound screening in the 3rd trimester. A de novo pathogenic variant c.2164G>A (p.G722S) was detected in sample P2, which has indications of micromelia skeletal dysplasia, mild dolichocephaly, small ventricular septal defect, and persistent right umbilical vein in the ultrasound screening in the 3rd trimester. Both variants were confirmed by Sanger sequencing using invasive or postnatal specimens. No pathogenic variants were detected among 45 pregnant women without target conditions.

The gestational ages at the time of sampling ranged from 10 to 40 weeks. The fetal fractions as calculated by the SNP-based assay ranged from 4.5 to 30%. In the analysis of the 30 genes, true positive calls are defined as the detection of either the paternal allele in the probands (i.e., when mother is homozygous for the reference allele and father is homozygous for the alternative allele) or de novo changes not detected in either the maternal or paternal samples but only present in the maternal plasma cell-free DNA. De novo variants identified were confirmed by a secondary assay (amplicon-based NGS) using the maternal cell-free DNA and/or Sanger sequencing using invasive or postnatal specimens. Five hundred fifty four true positive calls were detected in the 76 plasma samples. True negative calls are defined as the reference DNA sequence detected in both parents and the cell-free plasma DNA. For the true negatives (both parents are homozygous for the reference allele), over eight million nucleotides in the 30 genes of interest were accurately detected in the 76 samples. (Table 2)

TABLE 2
Summary of Sensitivity and Specificity
of Calls in 30 Genes (76 trios)

| | | |
|---|---|---|
| True Positive Calls | TP | 543 |
| True Negative Calls | TN | 7938342 |
| False Positive Calls | FP | 7 |
| False Positive Calls with 2^{nd} assay | — | 0 |
| False Negative Calls | FN | 0 |
| Sensitivity | TP/(TP + FN) | 100.0% |
| Specificity | TN/(TN + FP) | 100.0% |
| Positive Predictive Value | TP/(TP + FP) | 98.7% |
| Negative Predictive Value | TN/(TN + FN) | 100.0% |

False calls occurred in the primary capture-based NGS assay when both parents were homozygous for the reference alleles but the maternal cell-free plasma DNA showed a non-reference allele. An important distinction between de novo changes and false calls in the primary capture-based NGS assay was that all of the false positive could not be confirmed by our secondary assay using an amplicon-based NGS method. A total of seven analytical false positives were detected from five patients in the primary assay with relatively low fetal fractions (4.5, 4.7, 5.5, 6.0, and 8.6%). None of the false positives were detected using the amplicon-based NGS confirmatory test. Note that these variants were not considered pathogenic or likely pathogenic and they were only considered as analytical false positives for the primary assay. False negative calls were defined as DNA changes (either inherited paternal changes or de novo changes) that should have been present but were not detected in the cell-free plasma DNA. There were no false negatives in the genes of interest and in the SNPs across the genome.

Clinical Studies in Pregnancies with Abnormal Ultrasound Findings or Known Paternal Pathogenic Variants

In the clinical validation and initial offering of this newly developed single gene NIPT test, 101 consecutive samples were analyzed that include those with or without abnormal ultrasound findings, and those with known paternal history of conditions related to our 30-gene panel. Among them, 12 yielded positive results (11.9%) with at least a pathogenic or likely pathogenic variant identified. The gestational age in these samples ranged from 10 to 35 weeks with fetal fraction 4.9-20.4%. Twelve positive samples had nine de novo and three paternally inherited pathogenic variants, which included five cases with pathogenic variants in FGFR3, two in COL1A1, two in COL1A2 and one in each of NIPBL, PTPN11 and RIT1.

TABLE 3
Summary of Positive Cases

| | | Results | | | |
|---|---|---|---|---|---|
| | Fetal | | | Allele | |
| Sample | fraction | Gene | Mutation | frequency | Coverage |
| P1 | 19.7% | FGFR3 | c.1138G > A | 7.0% | 1,311 |
| | | | (p.G380R) | | |
| P2 | 18.2% | COL1A1 | c.2164G > A | 9.6% | 2,597 |
| | | | (p.G722S) | | |
| P3 | 9.0% | COL1A1 | c.3076C > T | 3.2% | 1,524 |
| | | | (p.R1026*) | | |
| P4 | 10.8% | FGFR3 | c.1948A > G | 4.20% | 1,588 |
| | | | (p.K650E) | | |
| P5 | 9.6% | RIT1 | c.229G > A | 3.2% | 5,665 |
| | | | (P.A77T) | | |
| P6 | 7.4% | NIPBL | c.1435C > T | 3.9% | 694 |
| | | | (p.R479*) | | |

Among five pregnancies (P13,P4,P12,P14,P10) with detected pathogenic variants in FGFR3 gene, three common variants, R248C, Y373C, K650E, and one rare pathogenic variant *807G were reported to cause thanatophoric dysplasia. All fetus have severe skeletal dysplasia such as short long bones in the 2nd trimester (14-22 weeks). In the first sample with detected pathogenic variant R1026* in the COL1A1 gene (P3), mother had normal ultrasound at the end of the first trimester. P3 has a paternal history of Osteogensis Imperfecta with the same pathogenic variant in the COL1A1 gene. In the second sample with novel likely pathogenic variant G296A in the COL1A1 gene (P4), the mother had abnormal ultrasound such as skeletal dysplasia and micromelia in the 3rd trimester. The same variant was detected in father's genomic DNA, who has short femus, shor humeri, and large forhead. In the other two patients with severe type of skeletal dysplasia (P7, P9), two de novo pathogenic variants, G835S and G895D, were detected in the COL1A2 gene. A de novo pathogenic variant A77T in the RIT1 gene was found in P5 with pleural effusion, hydrops, cystic hygroma and small low-set ears. There was a pathogenic variant R479* in the NIPBL gene in P6 which demonstrated symmetric growth restriction, possible diffuse skin thickening/edema, a duplicated right collecting system in the fetal kidneys, and a prominent philtrum in ultrasound screening. Both variants were confirmed by Sanger sequencing using fetal DNA, A77T from invasive assay and R479* from products of conception. In sample 11, the pathogenic variant Y279C in the PTPN11 gene was detected in both plasma and paternal genomic DNA, which is consistent with the family history of noonan syndrome disorders.

All pathogenic/likely pathogenic variants were confirmed by a different assay using cfDNA extracted from the second streak tube, four of 12 cases also confirmed by direct sequencing of fetal DNA from invasive, product of conception, or postnatal testing. A pathogenic variant, G835S of COL1A2 gene, was detected at very low percentage (˜1%) in maternal genomic DNA extracted from white blood cells of sample P9, which is also confirmed by a secondary assay. Since the inventors have not detected contribution of fetal allele in 22 loci when fetal inherited paternal only alleles in maternal genomic DNA, it is unlikely the mutant is from fetal cell or fetal cfDNA (Table 1). In conclusion, this appears to be a maternal mosaicism of the same mutant allele.

Significance of Certain Embodiments

The incidence of single gene disorders accounts for ˜0.36% of livebirths with congenital anomalies while chromosomal anomalies have an aggregated incidence of 0.18% underscoring the importance of providing practical screening for multiple Mendelian diseases (16). For instance, Noonan spectrum disorders (NSD) are a group of autosomal dominant diseases with accumulative prevalence at ˜1:1000 which have overlapping prenatal findings with trisomy 21, 18 and 13. Therefore, NSD is one of the diseases on differential diagnosis when prenatal ultrasound demonstrates increased nuchal translucence (17). In addition, FGFR3 related skeletal disorders are often suspected with shortened femur, humerus and/or frontal bossing found during late second trimester ultrasound screening (18). A common molecular etiology causing NSDs and FGFR3 related disorders are de novo pathogenic variants which arise at an increased rate with advanced paternal age (19). Unlike current NWT targeted for chromosomal aneuploidies usually associated with advanced maternal age, de novo point pathogenic variants are usually associated with advanced paternal age (20).

Random DNA changes can be introduced during NGS library preparation such as the PCR and sequencing processes, which can lead to an increase in background noise and potentially cause false positive results. To aid in separating the true DNA changes from artifacts, the inventors used unique DNA sequences referred to as UMI or “molecular barcodes”. Once plasma cell-free DNA is extracted from maternal blood, UMI are used to add unique labels to the cell-free DNA molecules before PCR. The technical process then includes library construction, target gene enrichment, and next-generation sequencing. During analysis of the sequence data, the random errors introduced during library preparation and sequencing steps are greatly suppressed by consolidation of the reads with the same UMI. A set of unique genome-wide SNPs was also used that is analyzed to calculate FF. Although not pathogenic, the SNPs inherited from the father are assessed in the cell-free DNA for every sample to ensure that these DNA changes are accurately identified and to provide an estimate of FF. Variety of approaches have been developed to detect FF in NIPT because the accuracy of the test is heavily dependent on the fetal DNA fraction (21), which is also observed in the increased number of analytical false positive calls in our approach for samples with low FF. The genetic markers on Y chromosome provided a very simple and accurate method in estimating FF for pregnancies with male fetuses (22). This approach is also used as gold standard in performance of other approaches such as cfDNA fragment size, differential methylation, and parental genotypes (reviewed in reference (21)). Our approach of FF estimation are based on combination of genome-wide SNP capture followed by deep sequencing, true molecular counting using UMI, and parental genotype. This approach generated highly consistent results with Y chromosome method or spike-in data.

Sequence data is carefully analyzed and variants from reference sequence are identified. The variants are carefully curated to determine if any meet criteria for classification as pathogenic or likely pathogenic. Analytical PPV is the probability that individuals with an identified variant truly have the variant. Note that this is different from clinical PPV, which is the probability that individuals with a positive screening test truly have the condition. Clinical PPV is dependent on the incidence of the disorder as well as large prospective studies using the screening method developed in this study.

Materials and Methods

Sample Collection

Study Design

This study has been performed in accordance with protocols approved by the Institutional Review Board for Human Subject Research at the Baylor College of Medicine (Houston, Tex.). A goal of the disclosure is to indicate if the fetus is at an increased risk for a genetic disorder that in at least some cases is followed up with invasive prenatal studies or neonatal studies. In one embodiment the disclosure provides a non-invasive prenatal screening test to detect de novo or paternally inherited pathogenic variants in 30 genes (as examples) frequently associated with dominant monogenic diseases.

Participants

223 maternal blood samples with gestational age of at least 10 weeks and singleton pregnancy were collected in BCT streak tube (two tubes of 8-10 ml for each), 200 of which had both parental blood/saliva available for trio-analysis. Clinical information such as abnormal ultrasound and historical genetic disorders of both parents were collected.

Test Methods

The plasma is separated through a two-step centrifugation process. The first step is to separate plasma with white blood cells by centrifugation at 1600 g for 15 minutes at 4° C. The second step is at 16,000 g for 10 minutes at 4° C. to remove cell debris. The cfDNA is extracted using QIAamp circulating nucleic acid extraction kit (Qiagen), and total genomic DNA is extracted from blood with a commercially available DNA isolation kit (Chemagen), according to the manufacturer's instructions. After cfDNA extraction individual molecules were tagged using custom developed Y-shaped adapter with unique molecular index to track individual molecules. The sample is then labeled by different sample index through PCR, purification, pooling for capture, washing and post-capture enrichment PCR, and sequencing on HiSeq 2500 (IIlumina, San Diego, Calif.) using 2×100 pair end sequencing. After demultiplexing, the NGS reads that share the same unique molecular index are grouped together to form sub groups. For each sub group, the consensus reads are consolidated to remove errors introduced during NGS library preparation or sequencing steps. The consolidated reads are used for alignment onto human genome hg 19 and followed by NGS data analysis. The variants were called by the NextGENe software version 2.3 (SoftGenetics, State College, Pa.). All minor alleles with <0.5% allele frequency were filtered out. The platform dependent errors are suppressed by a custom database with accumulation of all recurrent calls.

Fetal Fraction Estimation by Single Nucleotide Polymorphism (SNP) Analysis

A total of 153 SNPs spanning all chromosomes are included the target capture library. The SNPs inherited from parents in the fetal DNA can be analyzed to provide an estimate of FF. The FF was calculated based on two populations of informative loci. First, when the mother is homozygous for a reference allele and fetus inherits an alternative allele from the father at the same locus, the expected alternative allele fraction in the maternal cell-free DNA (MAFp) is half of the FF. Similarly, when the mother is homozygous for an alternative allele and fetus inherits paternal reference allele, the expected alternative allele fraction (MAFm) is 1 minus half of FF. The FF was then calculated based on the following formula, FF=MAFp+(1−MAFm).

Confirmation of Positive Results

All pathogenic and likely pathogenic variants are confirmed using an amplicon-based sequencing assay. The secondary method uses gene specific primers to enrich the targeted region using the second extraction of cfDNA as template. A second step PCR was used to add index and adapter sequences followed by deep sequencing (>20,000×) to confirm the variants in the cell-free DNA.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Norton M E, et al. (2015) Cell-free DNA analysis for noninvasive examination of trisomy. N Engl J Med 372 (17):1589-1597.
2. Chiu R W, et al. (2008) Noninvasive prenatal diagnosis of fetal chromosomal aneuploidy by massively parallel genomic sequencing of DNA in maternal plasma. Proceedings of the National Academy of Sciences of the United States of America 105 (51):20458-20463.
3. Chitty L S & Lo Y M (2015) Noninvasive Prenatal Screening for Genetic Diseases Using Massively Parallel Sequencing of Maternal Plasma DNA. Cold Spring Harb Perspect Med 5 (9):a023085.
4. Agatisa P K, et al. (2015) A first look at women's perspectives on noninvasive prenatal testing to detect sex chromosome aneuploidies and microdeletion syndromes. Prenat Diagn 35 (7):692-698.
5. Lo K K, et al. (2016) Limited Clinical Utility of Non-invasive Prenatal Testing for Subchromosomal Abnormalities. Am J Hum Genet 98 (1):34-44.
6. Chitty L S, et al. (2015) Non-invasive prenatal diagnosis of achondroplasia and thanatophoric dysplasia: next-generation sequencing allows for a safer, more accurate, and comprehensive approach. Prenat Diagn 35 (7):656-662.
7. You Y, et al. (2014) Integration of targeted sequencing and NIPT into clinical practice in a Chinese family with maple syrup urine disease. Genet Med 16 (8):594-600.
8. Lench N, et al. (2013) The clinical implementation of non-invasive prenatal diagnosis for single-gene disorders: challenges and progress made. Prenatal diagnosis 33 (6):555-562.
9. Lo Y M, et al. (2010) Maternal plasma DNA sequencing reveals the genome-wide genetic and mutational profile of the fetus. Sci Transl Med 2 (61):61ra91.
10. Chan K C, et al. (2016) Second generation noninvasive fetal genome analysis reveals de novo mutations, single-base parental inheritance, and preferred DNA ends. Proc Natl Acad Sci USA 113 (50):E8159-E8168.
11. Meacham F, et al. (2011) Identification and correction of systematic error in high-throughput sequence data. BMC bioinformatics 12:451.
12. Loman N J, et al. (2012) Performance comparison of benchtop high-throughput sequencing platforms. Nature biotechnology 30 (5):434-439.
13. Schirmer M, et al. (2015) Insight into biases and sequencing errors for amplicon sequencing with the Illumina MiSeq platform. Nucleic acids research 43 (6):e37.
14. Newman A M, et al. (2016) Integrated digital error suppression for improved detection of circulating tumor DNA. Nature biotechnology 34 (5):547-555.
15. Wang E, et al. (2013) Gestational age and maternal weight effects on fetal cell-free DNA in maternal plasma. Prenatal diagnosis 33 (7):662-666.
16. Baird P A, Anderson T W, Newcombe H B, & Lowry R B (1988) Genetic disorders in children and young adults: a population study. Am J Hum Genet 42 (5):677-693.
17. Allanson J E & Roberts A E (1993) Noonan Syndrome. GeneReviews(R), eds Pagon R A, Adam M P, Ardinger H H, Wallace S E, Amemiya A, Bean L J H, Bird T D, Ledbetter N, Mefford H C, Smith R J H, et al. Seattle (Wash.)).
18. Pauli R M (1993) Achondroplasia. GeneReviews(R), eds Pagon R A, Adam M P, Ardinger H H, Wallace S E, Amemiya A, Bean L J H, Bird T D, Ledbetter N, Mefford H C, Smith R J H, et al. Seattle (Wash.)).
19. Goriely A & Wilkie A O (2012) Paternal age effect mutations and selfish spermatogonial selection: causes and consequences for human disease. Am J Hum Genet 90 (2):175-200.
20. Glaser R L, et al. (2003) The paternal-age effect in Apert syndrome is due, in part, to the increased frequency of mutations in sperm. Am J Hum Genet 73 (4):939-947.
21. Peng X L & Jiang P (2017) Bioinformatics Approaches for Fetal DNA Fraction Estimation in Noninvasive Prenatal Testing. International journal of molecular sciences 18 (2).
22. Lo Y M, et al. (1998) Quantitative analysis of fetal DNA in maternal plasma and serum: implications for noninvasive prenatal diagnosis. American journal of human genetics 62 (4):768-775.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A non-invasive method of analyzing fetal DNA for one or more variants therein, comprising the steps of:
(a1) generating or providing a collection of circulating cell-free fetal DNA (cfDNA) fragments, each fragment comprising a first end ligated to a first adaptor and a second end ligated to a second adaptor, to produce fetal adaptor-ligated molecules,
wherein the first adaptor comprises a first strand and second strand having a complementary region there between that comprises a unique barcode and wherein the second adaptor comprises a first strand and second strand having a complementary region there between that comprises a unique barcode; and
(a2) generating or providing a collection of DNA fragments from the biological mother of the fetus and/or a separate collection of DNA fragments from the biological father of the fetus, wherein the fragments in the collection(s) comprise adaptor-ligated ends to produce maternal adaptor-ligated molecules and paternal adaptor-ligated molecules, respectively, wherein the adaptors each comprise a first strand and second strand having a complementary region there between;
(b) amplifying the fetal, maternal, and paternal adaptor-ligated molecules with primers complementary to a region of the respective adaptors to produce amplified adaptor-ligated molecules;
(c) enriching the amplified adaptor-ligated molecules for one or more target sequences of interest to produce enriched adaptor-ligated molecules;
(d) amplifying the enriched adaptor-ligated molecules;
(e) sequencing at least some of the enriched adaptor-ligated molecules; and
(f) analyzing the sequenced enriched adaptor-ligated molecules.

2. The method of claim 1, wherein the first and second adaptors each comprise a 5′ single-stranded end on their respective first strands in relation to their respective 3′ ends of their respective second strands.

3. The method of claim 1 or 2, wherein the unique barcode comprises 6 or more random nucleotides.

4. The method of claim 1, 2, or 3, wherein the adaptors in step (a2) lack a unique barcode.

5. The method of any one of claims 1-4, wherein the cfDNA fragments and/or the DNA fragments from the biological mother and biological father are subjected to end repair of the fragments and tailing of the fragment ends with a known nucleotide that is complementary to a nucleotide on the 3′ ends of the first strands of the adaptors.

6. The method of any one of claims 1-5, wherein the collection of DNA fragments from the biological mother and biological father are produced by fragmentation of genomic DNA from the biological mother and biological father, respectively.

7. The method of any one of claims 1-6, wherein in step (b) a primer binds a region of the respective adaptor-ligated molecules.

8. The method of claim 7, wherein in step (b) a primer binds the fetal adaptor-ligated molecules at a region that is 5′ to the unique barcode.

9. The method of any one of claims 1-8, wherein the enriching step comprises exposing the amplified adaptor-ligated molecules to probes that hybridize to a region of the amplified adaptor-ligated molecules.

10. The method of claim 9, wherein the probes target coding sequence.

11. The method of claim 9 or 10, wherein the probes are linked to a directly detectable agent or indirectly detectable agent.

12. The method of any one of claims 9-11, wherein the probes are linked to a first binding agent that binds to a second binding agent.

13. The method of claim 12, wherein the first binding agent is biotin and the second binding agent is avidin.

14. The method of claim 12 or 13, wherein the second binding agent is linked to a substrate.

15. The method of claim 14, wherein the substrate is a bead, plate, column, or well.

16. The method of any one of claims 1-15, wherein the target sequence is a coding sequence of a gene.

17. The method of any one of claims 1-16, wherein the target sequence is a coding sequence for an exon.

18. The method of any one of claims 1-17, wherein the target sequence(s) are of one or more genes associated with a monogenic Mendelian disorder.

19. The method of any one of claims 1-18, wherein the target sequences of interest are sequences from one or more genes in a collection of genes.

20. The method of claim 19, wherein the collection is a collection of sequences from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more different genes.

21. The method of any one of claims 1-20, wherein the sequencing step comprises next generation sequencing.

22. The method of any one of claims 1-21, wherein the analyzing step comprises comparing sequence between fetal sequenced enriched adaptor-ligated molecules and maternal sequenced enriched adaptor-ligated molecules and/or paternal sequenced enriched adaptor-ligated molecules.

23. The method of any one of claims 1-22 wherein the fetal DNA is obtained from the blood or plasma of the biological mother.

24. The method of any one if claims 1-23, wherein the variant is a de novo variant or is paternally-inherited.

25. The method of any one of claims 1-24, further comprising the step of assaying a fetal sample using an invasive and/or postnatal method for the fetus and/or biological mother.

26. The method of any one of claims 1-25, wherein at least one assay for the fetus during gestation had a determination of an abnormality or had a determination of a suspected abnormality.

27. The method of any one of claims 1-26, wherein the age of the biological father is greater than 45 years of age.

28. The method of any one of claims 1-27, wherein the variant comprises a single point mutation, insertion, deletion, or inversion.

29. The method of any one of claims 1-28, wherein the variant is associated with a monogenic Mendelian disorder.

30. The method of any one of claims 1-29, wherein the DNA of the biological father has a known variant associated with a disorder.

31. The method of any one of claims 1-30, wherein the variant is not aneuploidy.
